(19) United States
(12) Patent Application Publication
(10) Pub. No.: US 2016/0120818 A1
(43) Pub. Date: May 5, 2016

(54) PHARMACEUTICAL COMPOSITIONS COMPRISING VESICLES

(71) Applicants: GLAXOSMITHLINE BIOLOGICAL SA, Rixensart (BE); EXTERNAUTICS SPA, Siena (IT)

(72) Inventors: Guido Grandi, Siena (IT); Alberto Grandi, Siena (IT)

(21) Appl. No.: 14/766,202

(22) PCT Filed: Feb. 6, 2014
(86) PCT No.: PCT/EP2014/052370

§ 371 Date: Aug. 6, 2015

(30) Foreign Application Priority Data
Feb. 7, 2013 (EP) 13154463.7

(51) Int. Cl. A61K 9/50; A61K 9/127

(52) U.S. Cl. CPC A61K 9/5068; A61K 9/127

(57) ABSTRACT

The invention relates to pharmaceutical compositions comprising animal vesicles and bacterial vesicles, and to methods for preparing and using them. Animal vesicles and bacterial vesicles fuse to form immunogenic pharmaceutical compositions. The animal vesicular component provides a specific adaptive immune response and the bacterial vesicular component provides adjuventicity.

This application claims the benefit of the European application EP13154463.7 (filed Feb. 7, 2013), the complete contents of both of which are hereby incorporated herein by reference for all purposes.

### TECHNICAL FIELD

This invention relates to pharmaceutical compositions comprising animal vesicles and bacterial vesicles, methods for preparing said compositions, and uses thereof.

### BACKGROUND ART

Cancer is a major global cause of morbidity and mortality, which is expected to become increasingly prevalent in the coming decades. Conventional treatments for cancer include chemotherapeutic drugs, radiotherapy, and interventional surgery. Specific hormonal and antibody therapies, based on molecular expression profile of cancer cells, have also been developed for different cancer types (e.g. Herceptin, an anti-Her2 antibody for Her2 positive breast cancer).

Cancer vaccines have recently emerged as attractive alternative to conventional treatments for cancer because of their specificity, safety, and long-term immunological memory which is critical for controlling recurrences (Dougan et al., Annu Rev Immunol 2009; 27:83-117, PMID:19007331). Cervarix and Gardasil are successful examples of prophylactic vaccines showing efficacy for the prevention of cervical cancer. These vaccines, based on the delivery of antigens from cancer-causing human papillomavirus (HPV) variants, activate a strong antiviral immune response that consequently prevents the neoformation of HPV-induced cervical tumours.

In 2010, the Food and Drug Administration approved the use of a vaccine (Provenge) for the treatment of advanced prostate cancer. This is the first example of a therapeutic vaccine that stimulates the immune system against a self-antigen to promote killing of cancer cells. This vaccine is based on the use of activated dendritic cells pulsed with a prostate-specific protein (prostatic acid phosphatase-fusion protein) that prime the immune system to recognize and kill prostate cancer cells. However, the initial enthusiasm for this vaccine rapidly decreased owing to its moderate efficacy (4.1-month increase in survival time) and prohibitive costs (˜$93,000/dose/patient). Nevertheless, Provenge represents a milestone in cancer vaccine development and has opened new avenues in the personalized cancer vaccine therapy area.

However, most vaccines based on the delivery of tumour-associated antigens (TAAs) using different delivery vectors and/or formulated with a variety of adjuvants have led to disappointing results for four major reasons: (i) many TAAs are poorly immunogenic, since they are proteins that are either over-expressed (e.g. Her2) or carrying somatic mutations (e.g. RAS, p53) or translational modifications (e.g. MUC1); (ii) some TAAs are frequently highly expressed during foetal development (e.g. CEA) but not highly expressed in adults; (iii) TAAs, in particular the intracellular ones, have low antigenicity, because they are delivered inefficiently to antigen presenting cells (APC); (iv) TAAs are generally expressed in an immunosuppressive environment or in situations of TAAs established immune-tolerance caused by defective antigen presentation processes (e.g. lack of MHC I), absence of costimulatory molecules (e.g. lack of B7 molecules) and release of immunosuppressive factors (e.g. IL-10 and TFG).

New immunomodulatory reagents are under evaluation for the ability to reverse immunotolerance typical of advanced cancer states, and for the ability to increase the immune surveillance on cancer cells. Novel antigen delivery systems and adjuvants are also under development with the aim of enhancing the potency of cancer vaccines. These include dendritic cell activators and growth factors, vaccine adjuvants, T-cell stimulators and growth factors, genetically modified T cells, cytokines, agents to neutralize or inhibit suppressive cells. Adjuvants, including those used in the clinic, such as alum and MPL (Romanowski et al., Lancet 2009 Dec. 12; 374(9706):1975-85, PMID:19962185), and those used in the late stage of clinical development, tend to target the innate immune system for activation through pattern recognition receptors (PRR), such as TLRs.

Despite the recent advances in these fields, results from clinical studies of cancer vaccines (e.g. MyVax and FavId for the treatment of non-Hodgkin's lymphoma) are not yet satisfactory and there is still a demand for efficacious immunostimulatory molecules/vaccine delivery platforms able to overcome established tolerance in cancer patients and effectively raise T and B cells levels in vivo and to maintain T-cell number for prolonged periods of time. Enhanced immune responses are also desirable for treating diseases other than cancer, in particular, where patients may be immune-compromised, e.g. owing to infection, degenerative diseases, or old age.

Most antigens activate B cells using activated T helper (Th) cells, primarily Th1 and Th2 cells. Th1 cells secrete IFN-γ, which activates macrophages and induces the production of opsonizing antibodies by B cells. The Th1 response leads mainly to a cell-mediated immunity (cellular response), which protects against intracellular pathogens (invasive bacteria, protozoa and viruses). The Th1 response activates cytotoxic T lymphocytes (CTL), a sub-group of T cells, which induce death of cells infected with viruses and other intracellular pathogens. Natural killer (NK) cells are also activated by the Th1 response, these cells play a major role in the induction of apoptosis/killing of tumor cells, in cell infected by viruses and intracellular bacteria. On the other hand, Th2 cells generally induce a humoral (antibody) response critical in the defense against extracellular pathogens (helminthes, extracellular microbes and toxins).

The magnitude and type of Th response to a vaccine can be greatly modulated, depending on the adjuvant used for the antigen formulations. For instance, Alum, the most commonly used adjuvant in human vaccination, including vaccines against diphtheria-tetanus-pertussis, human papillomavirus and hepatitis vaccines (Marrack P et al., (2009). Nat Rev Immunol 9(4):287-93), mainly provokes a strong Th2 response, but is rather ineffective against pathogens that require Th1-cell-mediated immunity. Freund's Incomplete Adjuvant (IFA) induces a predominantly Th2 biased response with some Th1 cellular response. As forMF59®, it is a potent stimulator of both cellular (Th1) and humoral (Th2) immune responses (Ott G, (1995). Pharm Biotechnol 6: 277-96). Other adjuvants, essentially ligands for pattern recognition receptors (PRR), act by inducing the innate immunity, predominantly targeting the APCs and consequently influencing the adaptative immune response. Members of nearly all of the PRR families are potential targets for adjuvants. These include Toll-like receptors (TLRs), NOD-like receptors (NLRs), RIG-I-like receptors (RLRs) and C-type lectin receptors (CLRs). They signal through pathways that involve distinct adaptor molecules leading to the activation of different transcription factors. These transcription factors (NF-κB, IRF3) induce the production of cytokines and chemokines that play a key role in the priming, expansion and polarization of the immune responses.

As classical adjuvants induce strong Th2 response with little or no Th1 response, the current challenge is to develop adjuvants which induce a strong Th1 bias important for vaccines such as those against cancer, hepatitis, flu, malaria, and HIV. New adjuvants are being developed that are natural ligands or synthetic agonists for PRRs, either alone or with various formulations. PRR activation stimulates the production of pro-inflammatory cytokines/chemokines and type I IFNs that increase the host's ability to eliminate the pathogen. Thus, the incorporation of pathogens associated molecular patterns (PAMPs) in vaccine formulations can improve and accelerate the induction of vaccine-specific responses.

### DISCLOSURE OF THE INVENTION

The inventors have developed animal vesicle-bacterial vesicle complexes that could be used in pharmaceutical compositions, for example in vaccines. Specifically, the inventors have shown that exosome-OMV complexes form spontaneously when exosomes (animal vesicles) and OMVs (bacterial vesicles) are mixed together (see for example, Example 1), and the inventors hypothesize that stable fusion complexes are formed. Thus the invention provides a new platform for the development of highly immunogenic vaccines based on the co-delivery of animal vesicles and bacterial vesicles. The combined delivery of animal vesicles with bacterial vesicles represents a promising strategy for therapeutic vaccines to elicit an innate immune response by exploiting the major properties of the two components:
the strong adjuvanticity provided by the bacterial vesicle; and
the specific adaptive immune response against antigen(s) presented by the animal vesicle and associated with the targeted disease.

In addition the invention provides vesicles in particular exosome-OMV complexes which induce a strong Th1 bias important for vaccines such as those against cancer, hepatitis, flu, malaria, and HIV.

The invention is useful for any therapy where the presentation of a combination of antigens to the immune system of a patient may be beneficial. For example, the animal vesicles may present any disease-associated antigen, such as one or more TAA for cancer therapy, one or more pathogenic antigen for treatment of infection, or any other antigen or combination of antigens associated with other diseases, in particular for immune-compromised conditions and/or where strong potentiation of immunity is needed (e.g. in the elderly). Combined with the strong adjuvanting properties of the bacterial antigens of the bacterial vesicle, these disease-associated antigens presented by animal vesicles can provide an effective approach to vaccination.

Thus, the invention provides a pharmaceutical composition comprising: (a) an animal vesicle and (b) a bacterial vesicle. In some embodiments, the animal vesicles and bacterial vesicles are in a complex together e.g. by fusion of the lipid bilayers or by surface-molecule adhesion. In some embodiments the animal vesicles comprise disease-associated antigens, such as one or more TAA, one or more pathogen-associated antigen or one or more degenerative-disorder-associated antigen. In a preferred embodiment, the animal vesicles comprise TAAs. For example, in some embodiments, the animal vesicles are tumor-derived. In some embodiments, the animal vesicles are tumor-derived exosomes. In some embodiments the bacterial vesicles are outer membrane vesicles (OMVs), microvesicles (MVs [1]) or ‘native OMVs’ (‘NOMVs’). Thus, in one embodiment, the invention provides a pharmaceutical composition comprising tumour-derived exosomes and OMVs.

The invention also provides a method for preparing one or more complexes, wherein the method comprises a step of mixing (a) an animal vesicle with (b) a bacterial vesicle.

The invention also provides a complex comprising (a) an animal vesicle and (b) a bacterial vesicle. In some embodiments the complex is obtainable or obtained by a method of the invention. In some embodiments the complex is a fusion complex.

The invention also provides a method for preparing a pharmaceutical composition, wherein the method comprises a step of mixing (a) an animal vesicle with (b) a bacterial vesicle. In a preferred embodiment the pharmaceutical composition is an immunogenic composition.

Similarly, the invention provides a method for preparing a pharmaceutical composition, wherein the method comprises a step of mixing a first composition and a second composition, wherein the first composition comprises animal vesicles and the second composition comprises bacterial vesicles. After mixing, the process can include a step of permitting the vesicles from the first and second compositions to interact with each other, thereby to produce the pharmaceutical composition of the invention.

The invention also provides a composition for use in medicine, wherein the composition comprises (a) an animal vesicle and (b) a bacterial vesicle. This composition can be for use, for instance, in treating or preventing cancer e.g. where the animal vesicle includes a TAA.

The invention also provides a method for raising an immune response in a mammal, comprising administering a pharmaceutical composition of the invention to the mammal. This immune response can be an anti-tumour response e.g. where the animal vesicle includes a TAA.

The invention also provides the use of both an animal vesicle and a bacterial vesicle in the manufacture of a medicament, for example, for use in treating or preventing cancer.

The invention also provides a method for preparing a pharmaceutical composition, comprising steps of: (a) extracting a tumour cell from a mammalian subject; (b) obtaining a vesicle from the extracted tumour cell; and (c) mixing the obtained vesicle with a bacterial vesicle to provide the pharmaceutical composition. This composition can then be administered to the mammalian subject from whom the tumour cell was extracted in step (a).

The invention also provides a method for preparing a pharmaceutical composition, comprising steps of: (a) obtaining a vesicle from a tumour cell which was obtained from a mammalian subject; and (b) mixing the obtained vesicle with a bacterial vesicle to provide the pharmaceutical composition. This composition can then be administered to the mammalian subject from whom the tumour cell had been obtained before step (a).

### Animal Vesicles

An animal vesicle useful with the invention is an extracellular vesicle that is released from an animal cell. An animal vesicle is limited by a lipid bilayer that encloses biological molecules, and typically has a diameter of 20 to 1000 nm Various types of animal vesicle are known in the art, including membrane particles, membrane vesicles, microvesicles, exosome-like vesicles, exosomes, ectosome-like vesicles, ectosomes or exovesicles. Théry et al. (F1000 Biol Rep. 2011; 3: 15) provides a general review of exosomes and other similar secreted vesicles. The different types of animal vesicles are distinguished based on diameter, subcellular origin, their density in sucrose, shape, sedimentation rate, lipid composition, protein markers and mode of secretion i.e. following a signal (inducible) or spontaneously (constitutive). Four of the common animal vesicles and their distinguishing features are described in the following Table 1.

TABLE 1

| Animal | Diameter | | | | |
|---|---|---|---|---|---|
| vesicle | (nm) | Shape | Markers | Lipids | Origin |
| Micro- | 100-1000 | Irreg- | Integrins, | Phosphati- | Plasma |
| vesicles | | ular | selectins, | dylserine | membrane |
| | | | CD40 | | |
| | | | ligand | | |
| Exosome- | 20-50 | Irreg- | TNFRI | No lipid | MVB |
| like | | ular | | rafts | from |
| vesicles | | | | | other |
| | | | | | organelles |
| Exosomes | 30-100 | Cup | Tetraspanins | Cholesterol, | Multive- |
| | | shaped | (e.g. CD63, | sphingo- | sicular |
| | | | CD9), Alix, | myelin, | endo- |
| | | | TSG101, | ceramide, | somes |
| | | | ESCRT | lipid rafts, | |
| | | | | phosphati- | |
| | | | | dylserine | |
| Membrane | 50-80 | Round | CD133, | Unknown | Plasma |
| particles | | | no CD63 | | membrane |

Animal vesicles are thought to play a role in intercellular communication by acting as vehicles between a donor and recipient cell through direct and indirect mechanisms. Direct mechanisms include the uptake of the animal vesicle and its donor cell-derived components (such as proteins, lipids or nucleic acids) by the recipient cell, the components having a biological activity in the recipient cell. Indirect mechanisms include microvesicle-recipient cell surface interaction, and causing modulation of intracellular signalling of the recipient cell. Hence, animal vesicles may mediate the acquisition of one or more donor cell-derived properties by the recipient cell.

In some embodiments, the animal vesicle is a mammalian vesicle, i.e. it is from a mammalian cell. In some embodiments the animal vesicle is a human vesicle, i.e. it is from a human cell. Where the pharmaceutical composition is intended for administration to humans, human vesicles are preferred. The same origin/intent matching applies to other animals.

Any animal vesicle that is able to present disease associated-antigens to the immune system (e.g. due to having a tumour origin, being derived from an infected or mutated cell, or by other means) is useful in the context of the invention. Therefore, in some embodiments, the invention provides a pharmaceutical composition comprising a bacterial vesicle and an animal vesicle, wherein the animal vesicle includes at least one disease-associated antigen. In a further embodiment, the animal vesicle which includes at least one disease-associated antigen is a membrane particle, membrane vesicle, microvesicle, exosome-like vesicle, exosome, ectosome-like vesicle, ectosome or exovesicle. Exosomes and exosome-like particles are preferred animal vesicles of the invention because of their size, composition and ease of production.

In some embodiments the animal cell from which the animal vesicle is derived is a tumour cell. The tumour cell can be a primary tumour cell, or can be produced from a tumour cell e.g. by passaging, culture, expansion, immortalization, etc. Thus the tumour cell may be from a tumour in a cancer or pre-cancer patient, or may be from a tumour or cancer cell line. Tumour cells can provide animal vesicles which display TAAs. The tumour cell can be from a benign tumour or a malignant tumour.

In other embodiments, the animal cell from which the animal vesicle is derived is an infected cell, i.e. a cell that contains a pathogen.

In other embodiments, the animal cell from which the animal vesicle is derived is a mutated cell. For example, in some embodiments the mutated cell expresses mutant or misfolded proteins. In some embodiments, the mutated cell overexpresses one or more proteins. In some embodiments the mutant cell is involved in a degenerative disorder, such as a proteopathic disorder. In some embodiments, the animal cell is a central nervous system cell.

In some embodiments, the animal cell, such as the tumour cell, infected cell or mutated cell, may be autologous, i.e. from the patient that the pharmaceutical composition will be administered to.

Typically, a pharmaceutical composition for use as a vaccine for a particular cancer type will comprise animal vesicles derived from tumour/cancer cells of that particular cancer type. For example, a pharmaceutical composition for use in a prostate cancer vaccine typically comprises animal vesicles purified from prostate tumour/cancer cells. In this way, the animal vesicles comprise TAAs that stimulate an adaptive immune response to antigens present on the tumour/cancer cells to be treated/protected against. The same origin/intent matching applies to other diseases.

As shown in table 1, exosomes are nanoscale (30-100 nm) membrane vesicles formed by “inward/reverse budding” of the limiting membrane of the multivesicular bodies (MVBs) in the late endocytic compartment and released upon the fusion of MVB with the plasma membrane. Exosome secretion is observed from most cell types under both physiological and pathological conditions, particularly tumour cells and hematopoietic cells. Exosomes are easy to prepare and there are even commercially available kits for the purpose (e.g. the ExoQuick-TC kit from SBI).

Exosomes contain cytosolic and membrane proteins, as well as nucleic acid derived from the parental cells. The protein content is generally enriched for certain molecules, including targeting/adhesion molecules (e.g. tetraspanins, lactadherin and integrins), membrane trafficking molecules (e.g. annexins and Rab proteins), cytoskeleton molecules (e.g. actin and tubulin), proteins involved in MVB formation (e.g. Alix, Tsg101 and clathrin), chaperones (e.g., Hsp70 and Hsp90), signal transduction proteins (e.g. protein kinases, 14-3-3, and heterotrimeric G proteins) and cytoplasmic enzymes (e.g. GAPDH, peroxidases, and pyruvate kinases) (Yang C. & Robbins D. B. The role of tumor-derived exosomes in cancer pathogenesis. Clinical and Developmental Immunology, 2011, doi:10.1155/2011/842849). Other animal vesicles also contain various active molecules, such as those described above for exosomes. For example, membrane microvesicles and ectosomes have been shown to comprise cytokines, growth factor receptors, RNAs, and also metalloproteases.

Depending on their cellular origin the protein composition of animal vesicles can be enriched in specific proteins. For instance, tumour-derived animal vesicles usually contain TAAs expressed in the parental tumour cells such as melan-A, Silv, carcinoembryonic antigen (CEA), and mesothelin. Thus, cancer vaccine strategies have used tumour-derived exosomes as a source of TAAs to pulse DCs, resulting in the transfer of tumour antigens to DCs that were able to induce tumour-specific CD8+CTL response in mice (Wolfers J, Lozier A, Raposo G, et al. Tumor-derived exosomes are a source of shared tumor rejection antigens for CTL cross-priming. Nature Medicine. 2001; 7(3):297-303) and humans (Bu N, Wu H, Sun B, et al. Exosome-loaded dendritic cells elicit tumor-specific CD8(+) cytotoxic T cells in patients with glioma. Journal of Neuro-Oncology. 104(3):659-667).

In some embodiments, the animal vesicle can be modified to comprise additional proteins or to increase or reduce the level of a protein of interest. Typically, the modification will be applied to the cell that the vesicle is derived from prior to obtaining vesicles from said cell. Methods of altering protein expression are well known in the art and include, for example, genetic modification, inhibition by small molecule inhibitors, enzymes or other inhibitory/activating proteins or peptides, and antisense technology (or other nucleic acid technologies). For example, an animal vesicle can be modified to contain high levels of proinflammatory factors (Yang C. & Robbins D. B. The role of tumor-derived exosomes in cancer pathogenesis. Clinical and Developmental Immunology, 2011, doi:10.1155/2011/842849), e.g. by subjecting the cell that the vesicle is derived from to stress conditions under which proinflammatory cytokine and/or Hsp70 levels increase. This can result in animal vesicles that can stimulate Th1-polarized immune responses. Alternatively, the parent cell may be modified to reduce the expression of immunosuppressive molecules, such as FasL, TRAIL or TGF-beta. Animal vesicles can also be modified by incorporation of additional immunogenic proteins e.g. fusion with the superantigen staphylococcal enterotoxin A (SEA) (Xiu F, Cai Z, Yang Y, Wang X, Wang J, Cao X. Surface anchorage of superantigen SEA promotes induction of specific antitumor immune response by tumor-derived exosomes. Journal of Molecular Medicine. 2007; 85(5):511-521).

The protein content of the animal vesicle preparations can be analysed by methods well-known in the art, including for example Western blot, confocal microscopy, proteomics, etc.

Depending on their cellular origin and mechanism of synthesis, the lipid composition of the exosomes may also vary. These differences can be detected by methods well known in the art. Exosome-specific nucleic acids (such as miRNAs) can also be monitored. Thus an exosome can be characterized by protein, lipid and nucleic acid composition.

### Bacterial Vesicles

Bacterial vesicles useful with the invention can be any proteoliposomic vesicle obtained by disruption of or blebbling from a Gram-negative bacterial outer membrane to form vesicles which retain antigens from the outer membrane. Thus the term includes, for instance, OMVs (sometimes called ‘blebs’), microvesicles (MVs [1]) and ‘native OMVs’ (‘NOMVs’ [2]).

Bacterial vesicles have a number of properties which make them attractive candidates for vaccine delivery platforms including: (i) strong immunogenicity, (ii) self-adjuvanticity, (iii) capability to interact with mammalian cells and be taken up through membrane fusion or cell attachment via adhesion-receptors, and (iv) the possibility of incorporating heterologous antigen expression by recombinant engineering.

MVs and NOMVs are naturally-occurring membrane vesicles that form spontaneously during bacterial growth and are released into culture medium. MVs can be obtained by culturing bacteria in broth culture medium, separating whole cells from the smaller MVs in the broth culture medium (e.g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Strains for use in production of MVs can generally be selected on the basis of the amount of MVs produced in culture e.g. refs. 3 & 4 describe Neisseria with high MV production.

OMVs are prepared artificially from bacteria, and may be prepared using detergent treatment (e.g. with deoxycholate), or by non-detergent means (e.g. see reference 5). Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc.) at a pH sufficiently high not to precipitate the detergent [6]. Other techniques may be performed substantially in the absence of detergent [5] using techniques such as sonication, homogenisation, microfluidisation, cavitation, osmotic shock, grinding, French press, blending, etc. Methods using no or low detergent can retain useful antigens [5]. Thus a method may use an OMV extraction buffer with about 0.5% deoxycholate or lower e.g. about 0.2%, about 0.1%, <0.05% or zero.

Bacterial vesicles can conveniently be separated from whole bacteria by filtration e.g. through a 0.22 μm filter. Bacterial filtrates may be clarified by centrifugation, for example high speed centrifugation (e.g. 20,000×g for about 2 hours). Another useful process for OMV preparation is described in reference 7 and involves ultrafiltration on crude OMVs, instead of high speed centrifugation. The process may involve a step of ultracentrifugation after the ultrafiltration takes place. A simple process for purifying bacterial vesicles is described in reference 8, comprising: (i) a first filtration step in which the vesicles are separated from the bacteria based on their different sizes, with the vesicles passing into the filtrate e.g. using a 0.22 μm microfiltration; and (ii) a second filtration step in which the vesicles are retained in the retentate e.g. using a 0.1 μm microfiltration. The two steps can both use tangential flow filtration.

Another useful process for OMV production is to mutate the bacteria so that it spontaneously releases vesicles into the culture medium. For example, for meningococcus, it is possible to inactivate the mltA gene in a meningococcus, as disclosed in reference 9, and these mutant bacteria spontaneously release vesicles into their culture medium.

OMVs are lipid bilayer nanoscale spherical particles (10-300 nm in diameter) naturally and constitutively released by Gram negative bacteria during growth. OMVs are generated through a “budding out” of the bacterial outer membrane and, consistent with this, they have a composition similar to that of the bacterial outer membrane, including lipopolysaccharide (LPS), glycerophospholipids, outer membrane proteins, and periplasmic components (Mashburn-Warren and Whiteley, 2008; 2005). It has been proposed that OMV release is an essential step for bacteria to rapidly adapt to variations of the external environment. In addition, many other functions have been attributed to OMVs, including toxin and virulence factors delivery to host cells, inter species and intra species cell-to-cell cross-talk, biofilm formation, genetic transformation and defense against host immune responses.

Like their parent bacterial cells, OMVs activate the human immune system: LPS and outer membrane porins are part of the heterogeneous complex presented to the innate immune system as pathogen-associated molecular patterns (PAMPs). Pattern recognition receptors (PRRs) like Toll-like receptors (TLRs) present on the surface of host phagocytitic cells recognize LPS and other PAMPs and drive the inflammatory response in conjunction with the complement system (Amano et al., 2010; Beutler et al., 2003; Blander and Medzhitov, 2006; Schnare et al., 2001; Schnare et al., 2006). Furthermore, the fact that PAMPs immune potentiators are co-delivered with protective antigens through the OMVs internalization processes described above explains why OMVs are so effective in engendering protective immunity.

The content of OMVs or the intact OMVs can be taken up into mammalian cells by membrane fusion or through cell attachment via adhesion-receptors with vesicles using the same host receptors as bacteria (Ellis and Kuehn, 2010; Ellis et al., 2010; Kuehn and Kesty, 2005; Parker et al., 2010). The adherence of OMVs to host cells occurs both in vivo and in vitro. OMVs have also been detected in infected human tissues (Brown and Hardwidge, 2007; Kulp and Kuehn, 2010; Lee et al., 2008; Lindmark et al., 2009). The heat-labile enterotoxin (LT) produced by Enterotoxigenic E. coli (ETEC) is an example of an active toxin that can be delivered by OMVs to host cells (Brown and Hardwidge, 2007).

As mentioned above, it is also possible to incorporate heterologous antigens into bacterial vesicles, such as OMVs (Gorringe et al., 2009; O'Dwyer et al., 2004; Roy et al., 2010). For instance, Schroeder and Aebischer (2009) prepared recombinant OMVs from Salmonella carrying Leishmania antigens fused to C-terminal domains of an E. coli autotransporter that spontaneously integrates into the OM. The researchers found that subcutaneous injections of the recombinant vesicles boosted vaccine immune responses in mice, which were orally immunized with a live Salmonella vaccine, by up to 40 fold. Studies have also shown that heterologous proteins from other Gram-negative bacteria, and more recently, from a Gram-positive microbe can be incorporated into nascent OMVs by fusion with periplasmic or outer membrane proteins (Ashraf et al., 2011; Muralinath et al., 2011).

Although most clinical experience with vesicle-based vaccines is based on meningococcus, vesicle-based vaccines are also known for other Gram-negative bacteria.

Thus the vesicles may be from a species in any of genera Escherichia, Shigella, Neisseria, Moraxella, Bordetella, Borrelia, Brucella, Chlamydia Haemophilus, Legionella, Pseudomonas, Yersinia, Helicobacter, Salmonella, Vibrio, etc. For example, the vesicles may be from Bordetella pertussis, Borrelia burgdorferi, Brucella melitensis, Brucella ovis, Chlamydia psittaci, Chlamydia trachomatis, Moraxella catarrhalis, Escherichia coli (including extraintestinal pathogenic strains), Haemophilus influenzae (including non-typeable stains), Legionella pneumophila, Neisseria gonorrhoeae, Neisseria meningitidis, Neisseria lactamica, Pseudomonas aeruginosa, Yersinia enterocolitica, Helicobacter pylori, Salmonella enterica (including serovar typhi and typhimurium), Vibrio cholerae, Shigella dysenteriae, Shigella flexneri, Shigella boydii or Shigella sonnei, etc.

N. meningitidis OMVs have a proven safety record in humans and so may be a preferred choice. Another useful choice is E. coli vesicles, for example the BL21(DE3) strain (see Methods).

The vesicles can be prepared from a wild-type bacterium or from a modified bacterium e.g. a strain which has been modified to inactivate genes which lead to a toxic phenotype. For example, it is known to modify bacteria so that they do not express a native lipopolysaccharide (LPS), particularly for E. coli, meningococcus, Shigella, and the like. Various modifications of native LPS can be made e.g. these may disrupt the native lipid A structure, the oligosaccharide core, or the outer O antigen. Absence of O antigen in the LPS is useful, as is absence of hexa-acylated lipid A. Inactivation of enterotoxins is also known e.g. to prevent expression of Shiga toxin. If lipo-oligosaccharide (LOS) is present in a vesicle it is possible to treat the vesicle so as to link its LOS and protein components (“intra-bleb” conjugation). The vesicles may lack LOS altogether, or they may lack hexa-acylated LOS e.g. LOS in the vesicles may have a reduced number of secondary acyl chains per LOS molecule [10]. For example, the vesicles may be from a strain which has a lpxL1 deletion or mutation which results in production of a penta-acylated LOS. LOS in a strain may lack a lacto-N-neotetraose epitope e.g. it may be a lst and/or lgtB knockout strain. LOS may lack at least one wild-type primary O-linked fatty acid [11]. The LOS may have no a chain. The LOS may comprise GlcNAc-Hemphosphoethanolamine-KDO₂-Lipid A [12]. Bacteria can also be modified to reduce or knock-out expression of Tol-Pal. The Tol-Pal complex is a supramolecular machine in Gram-negative bacteria that spans the periplasm and is composed of both membrane and soluble proteins. The assembly is required for virulence in pathogenic organisms such as Vibrio cholerae, Pseudomonas aeruginosa and Salmonella typhimurium. Thus, in a preferred embodiment, the bacterial vesicles do not comprise a functional Tol-Pal system. As mentioned above, bacteria can also be modified by inactivation of the mltA gene.

Bacteria can be modified to have up-regulated antigens or expression of foreign antigens (i.e. antigens not native to the particular bacterial strain). As a result of this modification, vesicles prepared from modified bacteria contain higher levels of the up-regulated/foreign antigen(s). The increase in expression in the vesicles (measured relative to a corresponding wild-type strain) of the up-regulated antigen is usefully at least 10%, measured in mass of the relevant antigen per unit mass of vesicle, and is more usefully at least 20%, 30%, 40%, 50%, 75%, 100% or more.

Suitable recombinant modifications which can be used to cause up-regulation of an antigen include, but are not limited to: (i) promoter replacement; (ii) gene addition; (iii) gene replacement; or (iv) repressor knockout. In promoter replacement, the promoter which controls expression of the antigen's gene in a bacterium is replaced with a promoter which provides higher levels of expression. For instance, the gene might be placed under the control of a promoter from a housekeeping metabolic gene. In other embodiments, the antigen's gene is placed under the control of a constitutive or inducible promoter. Similarly, the gene can be modified to ensure that its expression is not subject to phase variation. Methods for reducing or eliminating phase variability of gene expression in meningococcus are disclosed in reference 13. These methods include promoter replacement, or the removal or replacement of a DNA motif which is responsible for a gene's phase variability. In gene addition, a bacterium which already expresses the antigen receives a second copy of the relevant gene. This second copy can be integrated into the bacterial chromosome or can be on an episomal element such as a plasmid. The second copy can have a stronger promoter than the existing copy. The gene can be placed under the control of a constitutive or inducible promoter. The effect of the gene addition is to increase the amount of expressed antigen. In gene replacement, gene addition occurs but is accompanied by deletion of the existing copy of the gene (see reference 14). Expression from the replacement copy is higher than from the previous copy, thus leading to up-regulation. In repressor knockout, a protein which represses expression of an antigen of interest is knocked out. Thus the repression does not occur and the antigen of interest can be expressed at a higher level. Promoters for up-regulated genes can advantageously include a CREN [15].

A modified strain will generally be isogenic with its parent strain, except for a genetic modification. As a result of the modification, expression of the antigen of interest in the modified strain is higher (under the same conditions) than in the parent strain. A typical modification will be to place a gene under the control of a promoter with which it is not found in nature and/or to knockout a gene which encodes a repressor.

In embodiments where antigen is up-regulated, various approaches can be used e.g. introduction of a gene expressing the antigenic protein of interest under the control of an IPTG-inducible promoter. The promoter may include a CREN.

The invention may be used with mixtures of vesicles from different strains (see, for example, ref. 16).

### Complexes

The inventors have shown that the animal vesicles and bacterial vesicles described above co-localise when mixed in solution, such as PBS. The inventors hypothesise that they form stable complexes.

Thus, in some embodiments, the invention provides a complex comprising an animal vesicle and a bacterial vesicle. In some embodiments the complex comprises a single animal vesicle and a single bacterial vesicle. In some embodiments, the complex comprises two or more animal vesicles (of the same type) and/or two or more bacterial vesicles (of the same type). For example, in some embodiments the complex comprises animal vesicle(s) and bacterial vesicle(s) in a ratio by number of vesicles of 1:1, 1:2, 1:3, 1:4 or more, or 2:1, 3:1, 4 or more:1.

Similarly, in some embodiments, an immunogenic composition of the invention comprises an animal vesicle and a bacterial vesicle, wherein the animal vesicle and bacterial vesicle are in a complex.

In some embodiments, the complex is formed by fusion of the two lipid bilayers, i.e. fusion of the animal vesicle lipid bilayer with the bacterial vesicle lipid bilayer. In some embodiments, the fusion results in a single closed lipid bilayer. Thus, in some embodiments the invention provides a “fusion complex” comprising: (i) bacterial antigens associated with adjuvanticity, optionally including PAMPs; and (ii) disease-associated antigens, such as TAAs.

As mentioned above exosomes can be characterized by protein, lipid and nucleic acid composition, which is dependent upon their cell of origin. These differences can be detected and used to distinguish exosomes from OMVs in the fusion. Thus, in some embodiments the fusion complex comprises animal glycoforms, animal lipids, animal nucleic acids and/or animal outer-membrane proteins, for example derived from the animal vesicles. The fusion complex is typically a vesicle with a lipid bilayer, optionally a single closed lipid bilayer (which may represent complete fusion of the two or more lipid bilayers).

In another embodiment, the complex is formed by surface attachment of protein and/or carbohydrate moieties on the two lipid bilayers, i.e. on the animal vesicle lipid bilayer and the bacterial vesicle lipid bilayer. For example, the surface attachment may be via adhesion-receptors.

In some embodiments, the complex is formed by a combination of fusion and surface attachment.

In some embodiments, there is no complex formation, i.e. the animal vesicle and bacterial vesicle are present in the pharmaceutical composition as separate components.

In some embodiments, the animal vesicles and bacterial vesicles are stored as separate components before being incorporated into a pharmaceutical composition. The components can be combined before, after or at the same time as administration to the animal. Thus, the separate components may be administered to an animal as separate components, typically simultaneously or sequentially. Alternatively, the separate components can be co-adminstered to the animal, for example using a dual-chambered syringe or a mixing syringe.

Co-localisation of the animal vesicles and bacterial vesicles can be determined by labelling the animal vesicles and bacterial vesicles (e.g. using a different fluorescent label for each), mixing the animal vesicles and bacterial vesicles together (e.g. in PBS) and observing the vesicles under a microscope (e.g. a laser-scanning confocal microscope).

### Disease-Associated Antigens

In general the animal vesicle comprises disease-associated antigens, for stimulating an immune response against the particular disease of interest.

Thus, in some embodiments, the animal vesicles comprise at least one disease-associated antigen. In some embodiments the at least one disease-associated antigen is a TAA, pathogen-associated antigen, or a degenerative disorder-associated antigen.

Some pathogens have been shown to express some of their antigens on the surface of the infected cells of the patient. Therefore, animal vesicles, such as exosomes, derived from these infected cells would also contain pathogen-associated antigens. In some embodiments the pathogen-associated antigen may be associated with a particular virus, bacterium, fungus, protozoa or a parasite. In a preferred embodiment the pathogen is an intracellular pathogen, i.e. a pathogen capable of growing and reproducing inside the cells of a host. Bacterial examples include but are not limited to Francisella tularensis, Listeria monocytogenes, Salmonella, Brucella, Legionella, Mycobacterium, Nocardia, Rhodococcus equi, Yersinia, Neisseria meningitidis, Chlamydia, Rickettsia, Coxiella, Mycobacterium, such as Mycobacterium leprae and Treponema pallidum. Fungal examples include but are not limited to Histoplasma capsulatum, Cryptococcus neoformans and Pneumocystis jirovecii. Examples of protozoa include but are not limited to Apicomplexans (e.g. Plasmodium spp., Toxoplasma gondii and Cryptosporidium parvum) and Trypanosomatids (e.g. Leishmania spp. and Trypanosoma cruzi).

Degenerative disorders include but are not limited to Amyotrophic Lateral Sclerosis (ALS), a.k.a., Lou Gehrig's Disease, Alzheimer's disease, Parkinson's Disease, Multiple system atrophy, Niemann Pick disease, Atherosclerosis, Progressive supranuclear palsy, Cancer, Essential tremor, Tay-Sachs Disease, Diabetes, Heart Disease, Keratoconus, Inflammatory Bowel Disease (IBD), Prostatitis, Osteoarthritis, Osteoporosis, Rheumatoid Arthritis, Huntington's Disease, Chronic traumatic encephalopathy and Chronic Obstructive Pulmonary Disease (COPD). In a further embodiment, the degenerative disorder is a proteopathic disease, in which certain proteins become structurally abnormal, and thereby disrupt the function of cells, tissues and organs of the body. Proteopathic diseases include but are not limited to Alzheimer's disease, Parkinson's disease, prion disease, type 2 diabetes, amyloidosis. Any of these degenerative disorders may have antigens associated with them the fall within the term “degenerative disorder-associated antigen”. For example, aggregating proteins of proteopathic diseases (see Table 2) or fragments thereof are examples of degenerative disorder-associated antigens.

TABLE 2

| | Major aggregating protein |
|---|---|
| Proteopathy | (disease-associated antigen) |
| Alzheimer's disease | Amyloid β peptide (Aβ); |
| | Tau protein (see tauopathies) |
| Cerebral β-amyloid angiopathy | Amyloid β peptide (Aβ) |
| Retinal ganglion cell degeneration | Amyloid β peptide (Aβ) |
| in glaucoma | |
| Prion diseases (multiple) | Prion protein |
| Parkinson's disease and other | α-Synuclein |
| synucleinopathies (multiple) | |
| Tauopathies (multiple) | Microtubule-associated protein |
| | tau (Tau protein) |
| Frontotemporal lobar degeneration | TDP-43 |
| (FTLD) (Ubi+, Tau−) | |
| FTLD-FUS | Fused in sarcoma (FUS) protein |
| Amyotrophic lateral sclerosis (ALS) | Superoxide dismutase, TDP-43, |
| | FUS |
| Huntington's disease and other triplet | Proteins with tandem glutamine |
| repeat disorders (multiple) | expansions |
| Familial British dementia | ABri |
| Familial Danish dementia | ADan |
| Hereditary cerebral hemorrhage with | Cystatin C |
| amyloidosis (Icelandic) (HCHWA-I) | |
| CADASIL | Notch3 |
| Alexander disease | Glial fibrillary acidic protein |
| | (GFAP) |
| Seipinopathies | Seipin |
| Familial amyloidotic neuropathy, | Transthyretin |
| Senile systemic amyloidosis | |
| Serpinopathies (multiple) | Serpins |
| AL (light chain) amyloidosis | Monoclonal immunoglobulin |
| (primary systemic amyloidosis) | light chains |
| AH (heavy chain) amyloidosis | Immunoglobulin heavy chains |
| AA (secondary) amyloidosis | Amyloid A protein |
| Type II diabetes | Islet amyloid polypeptide |
| | (IAPP; amylin) |
| Aortic medial amyloidosis | Medin (lactadherin) |
| ApoAI amyloidosis | Apolipoprotein AI |
| ApoAII amyloidosis | Apolipoprotein AII |
| ApoAIV amyloidosis | Apolipoprotein AIV |
| Familial amyloidosis of the | Gelsolin |
| Finnish type (FAF) | |
| Lysozyme amyloidosis | Lysozyme |
| Fibrinogen amyloidosis | Fibrinogen |
| Dialysis amyloidosis | Beta-2 microglobulin |
| Inclusion body myositis/myopathy | Amyloid β peptide (Aβ) |
| Cataracts | Crystallins |
| Retinitis pigmentosa with | rhodopsin |
| rhodopsin mutations | |
| Medullary thyroid carcinoma | Calcitonin |
| Cardiac atrial amyloidosis | Atrial natriuretic factor |
| Pituitary prolactinoma | Prolactin |
| Hereditary lattice corneal | Keratoepithelin |
| dystrophy | |
| Cutaneous lichen amyloidosis | Keratins |
| Mallory bodies | Keratin intermediate |
| | filament proteins |
| Corneal lactoferrin amyloidosis | Lactoferrin |
| Pulmonary alveolar proteinosis | Surfactant protein C (SP-C) |
| Odontogenic (Pindborg) | Odontogenic ameloblast- |
| tumor amyloid | associated protein |
| Seminal vesicle amyloid | Semenogelin I |
| Cystic Fibrosis | cystic fibrosis transmembrane |
| | conductance regulator (CFTR) |
| | protein |
| Sickle cell disease | Hemoglobin |
| Critical illness myopathy | Hyperproteolytic state of |
| (CIM) | myosin ubiquitination |

The animal vesicle may contain any combination of disease-associated antigens including any combination of pathogen-associated antigens, any combination of degenerative-disorder-associated antigens (including any combination of proteopathic antigens, as listed in Table 2), or any combination of TAAs.

### Tumour-Associated Antigens

TAAs (including tumour-specific antigens—TSAs) are proteins produced in tumour cells that have an abnormal structure and/or an abnormal expression pattern.

Oncofetal antigens are one class of tumour antigens. Examples are alphafetoprotein (AFP) and carcinoembryonic antigen (CEA). These proteins are normally produced in the early stages of embryonic development and disappear by the time the immune system is fully developed. Thus self-tolerance does not develop against these antigens.

Abnormal proteins are also produced by cells infected with oncoviruses, e.g. EBV and HPV. Cells infected by these viruses contain latent viral DNA which is transcribed and the resulting protein produces an immune response.

In addition to proteins, other substances like cell surface glycolipids and glycoproteins may also have an abnormal structure in tumour cells and could thus be targets of the immune system.

In some embodiments the animal vesicle of the invention comprise one or more TAA, wherein the one or more TAA is selected from: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumours; (b) mutated antigens, for example, p53 (associated with various solid tumours, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma); (e) prostate associated antigens such as PAP, PSA, PSMA, PSH-P1, PSM-P1, PSM-P2, associated with e.g., prostate cancer; (f) immunoglobulin idiotypes (associated with myeloma and B cell lymphomas, for example). In certain embodiments, the one or more TAA is selected from, but are not limited to, p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS, and the like. In some embodiments, the one or more TAA is selected from, but not limited to: melan-A, Silv, carcinoembryonic antigen (CEA), and mesothelin.

It is to be understood that the animal vesicle comprises any combination of 1, 2, 3, 4, 5, 6, 7, 8, 9, 10 or more TAAs selected from the TAAs listed above.

### The Pharmaceutical Composition

The pharmaceutical composition can include further components in addition to the animal vesicles and bacterial vesicles. These further components can include further immunogenic components and/or non-immunogenic components.

A pharmaceutical composition will usually include a pharmaceutically acceptable carrier, which can be any substance that does not itself induce the production of antibodies harmful to the patient receiving the composition, and which can be administered without undue toxicity. Pharmaceutically acceptable carriers can include liquids such as water, saline, glycerol and ethanol. Auxiliary substances, such as wetting or emulsifying agents, pH buffering substances, and the like, can also be present in such vehicles. A thorough discussion of suitable carriers is available in ref. 17.

The pH of the pharmaceutical composition is usually between 6 and 8, and more preferably between 6.5 and 7.5 (e.g. about 7). In some embodiments, stable pH in compositions of the invention may be maintained by the use of a buffer e.g. a Tris buffer, a citrate buffer, phosphate buffer, or a histidine buffer. Thus pharmaceutical compositions of the invention will generally include a buffer.

The pharmaceutical composition may be sterile and/or pyrogen-free. The pharmaceutical composition may be isotonic with respect to humans.

The invention also provides a container (e.g. vial) or delivery device (e.g. syringe) pre-filled with a pharmaceutical composition of the invention. The invention also provides a process for providing such a container or device, comprising introducing into the container or device a vesicle-containing composition of the invention.

Pharmaceutical compositions of the invention for administration to subjects are preferably vaccine compositions. Vaccines according to the invention may either be prophylactic (e.g. to prevent cancer) or therapeutic (e.g. to treat cancer). Pharmaceutical compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. The antigen content of compositions of the invention will generally be expressed in terms of the amount of protein per dose. The concentration of an antigen of interest in compositions of the invention may generally be between 10 and 500 μg/ml, preferably between 25 and 200 μg/ml, and more preferably about 50 μg/ml or about 100 μg/ml (expressed in terms of total protein in the composition).

The concentration of the animal and bacterial vesicles included in the pharmaceutical compositions will vary depending on a number of parameters including, for example the cell type from which the vesicle is derived. The concentration of animal vesicles in the compositions will generally be 10⁸ to 10⁹ vesicles per ml. Typically, the animal vesicles and bacterial vesicles will be mixed in equal quantities by moles. However, in some embodiments, depending on the levels of surface antigens, a greater proportion of animal vesicles or a greater proportion of bacterial vesicles will be present in the pharmaceutical composition. For example, in some embodiments the animal vesicles are mixed with the bacterial vesicles in a ratio by molar quantity of from 1:10 to 10:1, from 1:9 to 9:1, from 1:8 to 8:1, from 1:7 to 7:1, from 1:6 to 6:1, from 1:5 to 5:1, from 1:4 to 4:1, from 1:3 to 3:1, from 1:2 to 2:1 or 1:1.

Pharmaceutical compositions may include an immunological adjuvant. Thus, for example, they may include an aluminium salt adjuvant or an oil-in-water emulsion (e.g. a squalene-in-water emulsion).

Suitable aluminium salts include hydroxides (e.g. oxyhydroxides), phosphates (e.g. hydroxyphosphates, orthophosphates), (e.g. see chapters 8 & 9 of ref. 18), or mixtures thereof. The salts can take any suitable form (e.g. gel, crystalline, amorphous, etc.), with adsorption of antigen to the salt being preferred. The concentration of Al⁺⁺⁺ in a composition for administration to a subject is preferably less than 5 mg/ml e.g. ≦4 mg/ml, ≦3 mg/ml, ≦2 mg/ml, ≦1 mg/ml, etc. A preferred range is between 0.3 and 1 mg/ml. A maximum of 0.85 mg/dose is preferred.

Compositions of the invention may be prepared in various liquid forms. For example, the compositions may be prepared as injectables, either as solutions or suspensions. The composition may be prepared for pulmonary administration e.g. by an inhaler, using a fine spray. The composition may be prepared for nasal, aural or ocular administration e.g. as spray or drops, and intranasal vesicle vaccines are known in the art. Injectables for intramuscular administration are typical. Injection may be via a needle (e.g. a hypodermic needle), but needle-free injection may alternatively be used.

Compositions may include an antimicrobial, particularly when packaged in multiple dose format. Antimicrobials such as thiomersal and 2-phenoxyethanol are commonly found in vaccines, but it is preferred to use either a mercury-free preservative or no preservative at all.

Compositions may comprise detergent e.g. a Tween (polysorbate), such as Tween 80. Detergents are generally present at low levels e.g. <0.01%.

Compositions may include residual detergent (e.g. deoxycholate) e.g. from OMV preparation. The amount of residual detergent is preferably less than 0.4 μg (more preferably less than 0.2 μg) for every μg of vesicle protein.

If a composition includes LOS, the amount of LOS is preferably less than 0.12 μg (more preferably less than 0.05 μg) for every μg of vesicle protein.

Compositions may include sodium salts (e.g. sodium chloride) e.g. for controlling tonicity. A concentration of 10±2 mg/ml NaCl is typical e.g. about 9 mg/ml.

Effective dosage volumes can be routinely established, depending on the antigenicity of the composition. Typical human dose of the composition might be, for example about 0.5 ml e.g. for intramuscular injection (e.g. into the thigh or upper arm). Similar doses may be used for other delivery routes e.g. an intranasal vaccine for atomization may have a volume of about 100 μl or about 130 μl per spray, with four sprays administered to give a total dose of about 0.5 ml.

### Uses of the Invention

The invention also provides a complex or composition of the invention, for use in medicine, for example for use in treating or preventing a disease.

The invention also provides a method for treating or preventing a disease comprising administering a pharmaceutical composition of the invention to a mammal, preferably a human.

The invention also provides a method for raising an immune response in a mammal, comprising administering a pharmaceutical composition of the invention, preferably an immunogenic composition, to the mammal. Typically, the immune response is an antibody response. The antibody response is preferably a protective antibody response. The invention also provides compositions of the invention for use in such methods.

The invention also provides a method for protecting a mammal against a disease, such as cancer, comprising administering to the mammal a pharmaceutical composition of the invention.

The invention provides pharmaceutical compositions of the invention for use as medicaments (e.g. as immunogenic compositions or as vaccines). It also provides the use of animal vesicles and bacterial vesicles, optionally in complexes, in the manufacture of a medicament for treating or preventing disease in a mammal.

The disease may be, for example, (but is not limited to) a pathogenic infection (such as those listed elsewhere in the application), Amyotrophic Lateral Sclerosis (ALS), a.k.a., Lou Gehrig's Disease, Alzheimer's disease, Parkinson's Disease, Multiple system atrophy, Niemann Pick disease, Atherosclerosis, Progressive supranuclear palsy, Cancer, Essential tremor, Tay-Sachs Disease, Diabetes, Heart Disease, Keratoconus, Inflammatory Bowel Disease (IBD), Prostatitis, Osteoarthritis, Osteoporosis, Rheumatoid Arthritis, Huntington's Disease, Chronic traumatic encephalopathy and Chronic Obstructive Pulmonary Disease (COPD). In a further embodiment, the degenerative disorder is a proteopathic disease, in which certain proteins become structurally abnormal, and thereby disrupt the function of cells, tissues and organs of the body. Proteopathic diseases include but are not limited to Alzheimer's disease, Parkinson's disease, prion disease, type 2 diabetes, amyloidosis.

The cancer may be, for example, (but is not limited to), bronchogenic carcinoma, nasopharyngeal carcinoma, laryngeal carcinoma, small cell and non-small cell lung carcinoma, lung adenocarcinoma, hepatocarcinoma, pancreatic carcinoma, bladder carcinoma, colon carcinoma, breast carcinoma, cervical carcinoma, ovarian carcinoma, or lymphocytic leukaemias.

The vesicles selected for use in the present invention should be selected as vesicles containing appropriate antigens for raising an immune response for a particular disease. Appropriate vesicles could easily be selected by the skilled person. For example, to prepare a pharmaceutical composition or complex for use in treating cancer, the skilled person might select an animal vesicle, such as an exosome, derived from a tumour, and combine that animal vesicle with a bacterial vesicle capable of adjuvanting the immune response when administered to the cancer patient. Similarly, the vesicles should contain appropriate antigens for the disease of interest. For example, for the proteopathic disorders listed in Table 2, the skilled person might ensure that an antigen of the one or more corresponding listed aggregating proteins were included in the animal vesicle.

The mammal is preferably a human. The human may be an adult or a child. A vaccine intended for children may also be administered to adults e.g. to assess safety, dosage, immunogenicity, etc.

Efficacy of therapeutic treatment can be tested by monitoring infection or tumour progression after administration of the composition of the invention. Efficacy of prophylactic treatment can be tested by monitoring immune responses against immunogenic proteins in the vesicles or other antigens, for example TAAs, after administration of the composition Immunogenicity of compositions of the invention can be determined by administering them to test subjects and then determining standard serological parameters. These immune responses will generally be determined around 4 weeks after administration of the composition, and compared to values determined before administration of the composition. Where more than one dose of the composition is administered, more than one post-administration determination may be made. In general, pharmaceutical compositions of the invention comprising animal vesicles which include TAAs are able to induce serum anti-TAA antibody responses after being administered to a subject.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is about 0.5 ml.

The invention may be used to elicit systemic and/or mucosal immunity.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x+10%.

Unless specifically stated, a process comprising a step of mixing two or more components does not require any specific order of mixing. Thus components can be mixed in any order. Where there are three components then two components can be combined with each other, and then the combination may be combined with the third component, etc.

### BRIEF DESCRIPTION OF DRAWINGS

FIG. 1. OMVs-exosomes complexes. OMVs purified from E. coli ΔTolR were labeled by incubation with FM 4-64 dye and incubated with exosomes isolated from the EGFP-transfected HEK293T cell line. Exosome-OMV co-localisation was assessed by a laser scanning confocal microscope with 488 nm/543 nm laser lines. Upper panels of FIG. 1A: purified EGFP-exosomes and OMVs FM 4-64 dye scanned with laser lines 488 and 542 nm respectively. The original fluorescence signals of exosomes and OMVs is converted to a white and grey scale (EGFP-exosomes, dark grey spots; OMVs FM 4-64, white spots); merging of the two images shows that two of the three visible OMVs (spots 1 and 3) show both 488 and 542 nm fluorescence signals, indicating that these vesicles colocalise with exosomes. Lower panels of FIGS. 1B, 1C and 1D: co-localisation graph of the visible vesicles. The graphs represent the fluorescence intensity per micrometer of the OMV and exosome spots and show the overlapping signals between EGFP-exosomes and OMVs FM 4-64 (labeled as spots 1 and 3) (EGFP-exosomes, solid line; OMVs FM 4-64, dashed line)

FIG. 2. Western blot with an antibody raised against IFITM3, a protein known to be exosome-associated.

FIG. 3. Total IgGs elicited by the OMV+exosomes formulations against 5 exosome associated human proteins (CXCR4, EFFR, IFITM3, FOLH1, TFRC). Sera from mice immunized with OMV+exosomes and OMV alone were pooled and analyzed by ELISA on each recombinant proteins, as compared to pre-immune sera

FIG. 4. IgG1 and IgG2a antibody subclasses elicited by the OMV+exosomes against 5 exosome associated human proteins. Sera from mice immunized with OMV+exosomes, exosomes and OMV alone were pooled and analyzed by ELISA on each recombinant proteins.

### MODES FOR CARRYING OUT THE INVENTION

### Example 1

### Methods

### Exosome Purification and Analysis

The Hek293-EGFP stable clone was developed by stably transfecting HEK293-FLP in cells (Invitrogen) using a plasmid encoding the green fluorescent protein EGFP (pcDNA-EGF) under the manufacturer's conditions. Hek293-EGFP cells were cultured in DMEM 10% FBS at 37° C. with 5% CO₂. When cells were at 80-90% of confluence, the medium was replaced with fresh serum-free medium. After 24 hours we collected 10 ml of cell culture supernatant and exosomes were purified using the ExoQuick-TC kit (SBI), following the provider's protocol.

The quality of the exosomes preparations was analysed by western blot by confocal microscopy, using antibodies raised against a panel of proteins known to be associated with exosomes. Moreover, exosomes were stained with antibodies against tumour-associated antigens expressed by different cell lines and detected in exosomes (for example, see FIG. 2).

For Western blot, the exosomal pellet was resuspended in 20 ul of Laemmli loading, boiled 10′ and separated by SDS-PAGE and transferred onto PVDF membrane. The membrane was saturated with PBS+10% dry fat milk for 1 hour at RT. The membrane, incubated in PBS with 1% not fat dry milk and 0.05% Tween, were probed first with polyclonal at 1:1000 dilution, ON at 4° C. After three washes with PBS with 1% not fat dry milk and 0.05% Tween, was added the secondary antibody at 1:500 dilution for 1 hour at RT. After three other washes with PBS+0.05% Tween, the membrane was developed with ECL and detected with ChemiDoc. For confocal microscopy analysis, the Hek293-EGFP exosomes were isolated with Exoquick-Tc and observed under a laser-scanning confocal microscope with 488 nm laser line (LeicaSPS).

### OMV Preparation

Culture media of the BL21(DE3) ΔtolR mutant strain, lacking of a functional TolR gene, strains were filtered through a 0.22-μm-pore size filter (Millipore, Bedford, Mass.). The filtrates were clarified by centrifugation and subjected to high speed centrifugation (200,000×g for 2 hours), and the pellets containing the OMVs were washed with PBS and finally resuspended with PBS. OMVs were labeled with the FM 4-64 dye (Molecular probes) for confocal microscopy, using standard protocol procedures.

Alternatively, OMV can be prepared from BL21(DE3)ΔompA E. coli cells inoculated from fresh plate into 500 ml of LB (Luria Bertani broth)+Amp (100 ug/ml) and were incubated at 37° C. with shaking (200 r.p.m.) and growth. Bacteria culture were grown until at 37° C. the O.D.=1. At that point, culture media were filtered through a 0.22 μm pore-size filter (Millipore, Bedford, Mass.). The filtrates were subjected to high speed centrifugation (200,000×g for 90 min), and the pellets containing the OMVs were washed with PBS and finally resuspended with PBS (Berlanda et al. (2008) Mol Cell Proteomics 2008 March; 7(3):473-85).

### Generation of OMV-Exosomes Complexes

To demonstrate the capability of OMVs to interact with exosomes, colocalization studies were carried out. OMVs were labeled with the FM 4-64 dye, mixed with equal volume of EGFP-exosome preparation for 30 minutes at room temperature in PBS, diluted and plated on glass cover slips, and mounted with glycerol plastine. OMV-exosome complexes were visualized under a laser-scanning confocal microscope with 488 nm/543 nm laser lines (LeicaSPS).

### Results and Conclusions

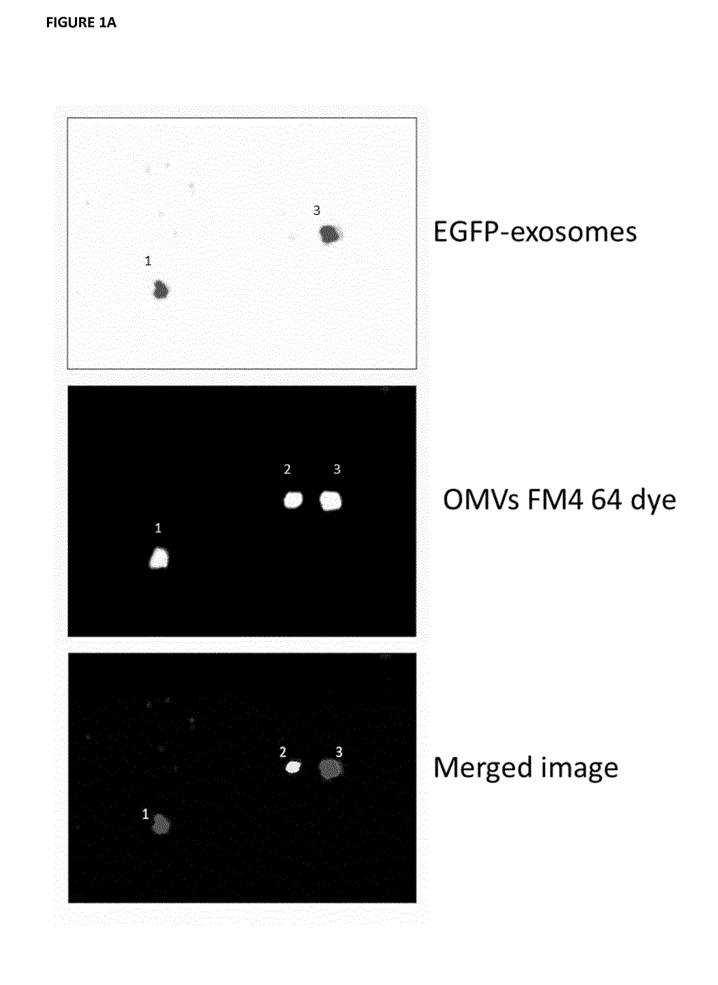

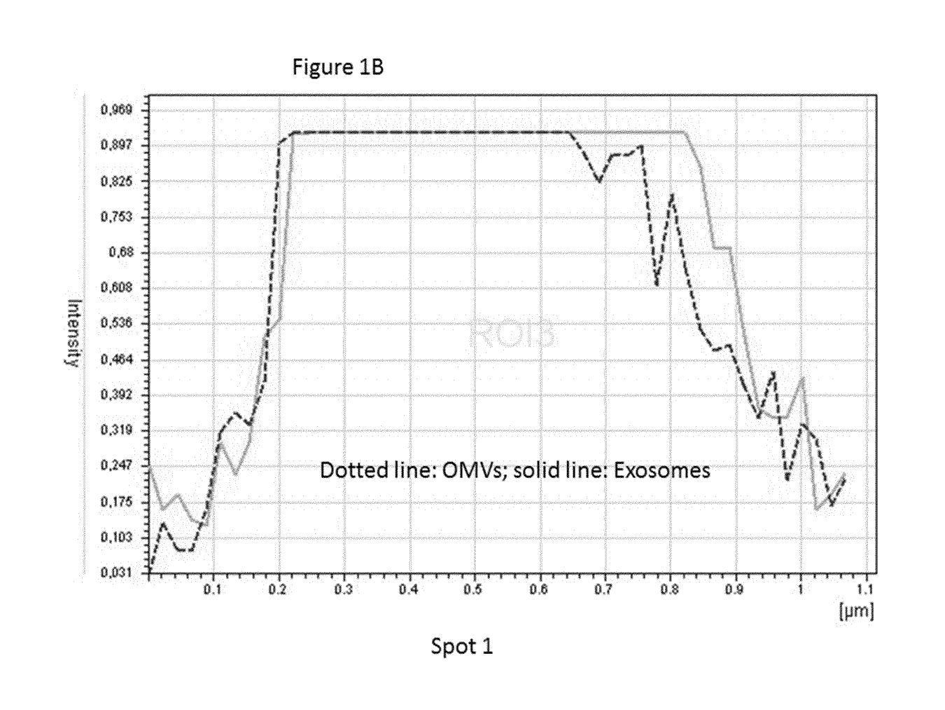

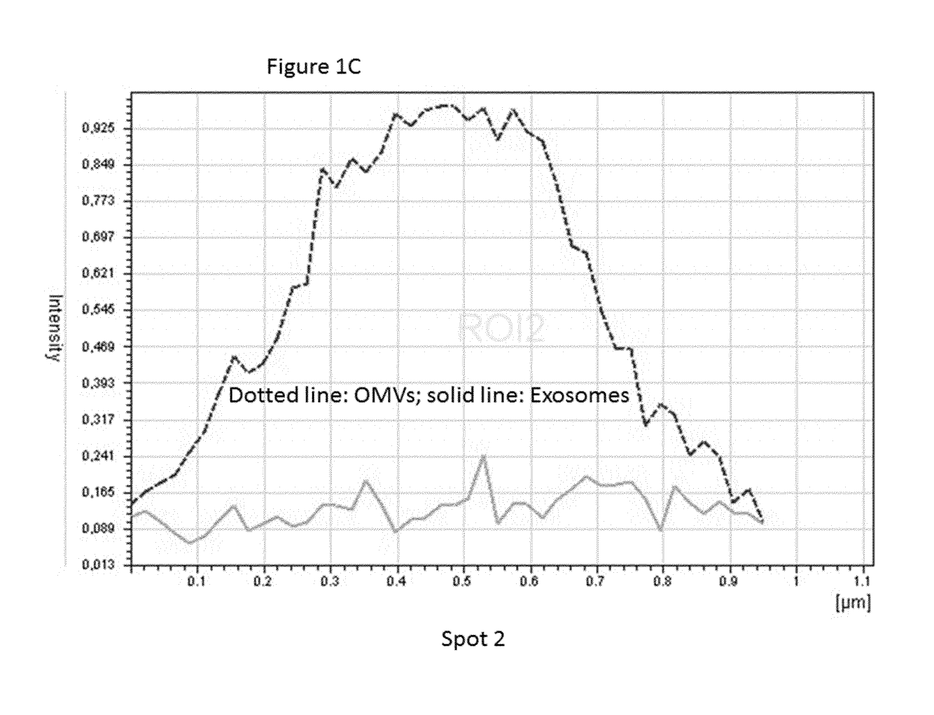

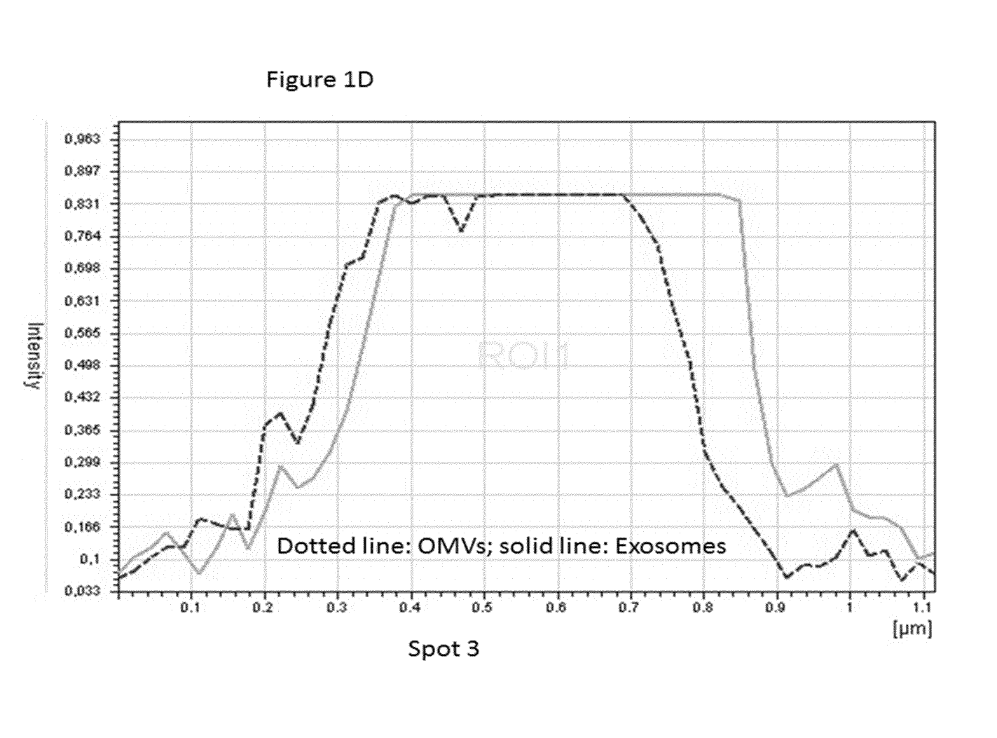

An analysis of EGFP-exosomes showed that they contain CD81, one of the most abundant exosomal proteins. Moreover, exosomes showed the presence of a panel of tumour-associated antigens that could be exploited for use in vaccines. OMVs were labeled with FM 4-64 dye and were incubated with purified EGFP-exosomes. Exosome-OMV co-localization was verified by a laser-scanning confocal microscope with 488 nm/543 nm laser lines. In this analysis, the presence of OMV—exosome complexes is revealed by overlapping 488 nm and 542 nm fluorescence signals, whereas distinct OMVs and exosomes show either of the two fluorescence signals. As shown in FIG. 1, when EGFP-exosomes and OMVs FM 4-64 were co-incubated, some OMV and exosome vesicles had both fluorescence signals, indicating that bacterial and mammalian vesicles co-localize (represented by light grey spots labeled as 1 and 3 in FIG. 1, panel A), and forms complexes.

### Example 2

### Methods

### OMV Preparation

OMV were prepared from BL21(DE3)ΔompA E. coli cells inoculated from fresh plate into 500 ml of LB (Luria Bertani broth)+Amp (100 ug/ml) and were incubated at 37° C. with shaking (200 r.p.m.) and growth. Bacteria culture were grown until at 37° C. the O.D.=1. At that point, culture media were filtered through a 0.22 μm pore-size filter (Millipore, Bedford, Mass.). The filtrates were subjected to high speed centrifugation (200,000×g for 90 min), and the pellets containing the OMVs were washed with PBS and finally resuspended with PBS (Berlanda et al. (2008). Mol Cell Proteomics 2008 March; 7(3):473-85).

### Preparation of Exosomes for Immunization Studies

For immunization studies, exosomes from cell culture supernatants were isolated by differential centrifugation as described by Raposo et al. (1996) Exp. Med. 183, 1161-1172. CD81 Briefly, 1×10⁸ HCT15 cells were cultured in DMEM-10% FCS until confluency in 18 175 cm² flasks until pre-confluence. For exosomes preparation, the culture medium was replaced with serum-free medium (PFHM-II Gibco-Life Technologies), cultured for 24 h and then centrifuged at 200×g for 10 min (pellet P1). The supernatant was collected and centrifuged twice at 500 g for 10 min (pellet P2). The second supernatant was sequentially centrifuged at 2,000×g twice for 15 min (pellet P3), once at 10,000×g for 30 min (pellet P4), and once at 70,000×g for 60 min (pellet P5), using a SW28 rotor (Beckman instruments, Inc.). The cellular pellet P1 was solubilized in 1 ml of C-RIPA buffer (50 mM Tris-Hcl pH7.5, 150 mM NaCl, 1% Nonidet P-40; 2 mM EGTA, 1 mM orthovanadate, 0.1% SDS, 0.5% Na-deoxycholate, 1 mM phenyl-methane-sulphonylfluoride, 10 μg/ml leupeptin, 10 μg/ml aprotinin) while each of the supernatant-derived pellets P2-P5 were solubilized in 0.5 l of the same buffer. After clarification, the protein concentration of each sample was determined by Bradford.

As quality controls of the exosomial preparation, 20 μg of P1 extract and 10 μg of P2-P5 extracts (corresponding to approximately 2×10⁵ and 2×10⁷ cells, respectively) were loaded on SDS-PAGE (4-12%) and analyzed by Western blot with antibodies targeting the exosomial marker CD81. Moreover, the presence the five proteins in exosomes was also assessed by Western blot. SDS-PAGE (4-12% gel) were electroblotted onto nitrocellulose membranes. Membranes were saturated for 1 h at room temperature, in blocking buffer composed of 1×PBS-0.1% Tween 20 (PBST) containing 10% dry milk. Then, the membrane were incubated with antigen-specific antibodies diluted 1:1000 in blocking buffer containing 1% dry milk and washed in PBST-1%. The secondary HRP-conjugated antibody (goat anti-mouse immunoglobulin/HRP, Perkin Elmer) was diluted 1:5000 in blocking buffer, and chemiluminescence detection was carried out using a Chemidoc-IT UVP CCD camera (UVP) and the Western Lightning™ Cheminulescence Reagent Plus (Perkin Elmer), according to the manufacturer's protocol.

### Immunizations

5/6 week old CD1 outbred female mice (5 mice per group) were immunised intra-peritoneally at days 1, 14 and 28 with either OMV (15 micrograms in 100 microliters) or the combination of OMV+exosomes (15 micrograms each, in 100 microliters) or exosomes alone (15 micrograms, in 100 microliters) formulated with an equal volume of Alum Hydroxide as adjuvant at the final concentration of 3 mg/ml. Two weeks after the last immunization mice were bled and sera from individual mice were pooled.

### ELISA Analysis

Total IgG titers elicited by immunizing mice with the combination of OMV+exosomes were tested on a panel of exosomial proteins were assayed by enzyme-linked immunosorbent assay (ELISA). Individual wells of micro-ELISA plates (Nunc Maxisorp) were coated with 1 μg of each recombinant protein in PBS (pH 7.4) at 4° C. overnight. The plates were washed, treated for 1 h at 37° C. with PBS-1% BSA, and 100 μl aliquots of anti-sera towards OMV, exosomes and OMV+exosomes, at different serial dilutions in PBS-0.1% Tween, were added to the wells. After incubation for 2 h at 37° C., plates were again washed and incubated for 1 h at 37° C. with alkaline-phosphatase conjugated goat anti-mouse IgG (Sigma) diluted 1:2500 in PBS-Tween.

For the detection of IgG2a and IgG1 subclasses, plates were incubated with alkaline-phosphatase conjugated goat anti-mouse IgG2a and IgG1 (Sigma), diluted at 1:4000 in PBS-Tween. Thereafter 100 μl of PNPP (Sigma) were added to the samples and incubated for 30 min. at room temperature. Optical densities were read at 405 nm and the sera-antibody titers were defined as the serum dilution yielding an OD value of 0.5.

### Results and Conclusions

### Marker Detection in HCT15-Derived Exosomes

The expression of a panel of exosome-associated proteins in HCT15 cells was verified by Western blot in total extract and/or the exosomial fraction of HCT15 cells. Exosomes were prepared by sequential differential centrifugations of the culture supernatants that yielded five centrifugation pellets, of which P1 represents the cellular pellets, P2-P4 are intermediate supernatant-derived pellets and P5 is the final exosome-enriched pellet. The pellets P1, (20 μg/lane, corresponding to approximately 1×10⁵ cells), P4 and the final exosome pellet P5 (10 μg/lane, corresponding to 2×10⁷ cells) were subjected to Western blot with antibodies raised against the exosomial marker CD81, and with polyclonal antibodies against 5 selected proteins, listed in the below Table A:

TABLE A

| | Description (GeneCards summary available at |
|---|---|
| Protein | http://www.genecards.org) |
| TFRC | TFRC (transferrin receptor). Diseases associated with TFRC |
| | include gastric adenosquamous carcinoma, and berger disease, |
| | and among its related super-pathways are Golgi Associated |
| | Vesicle Biogenesis and Insulin receptor recycling. |
| FOLH1 | FOLH1 gene encodes a type II transmembrane glycoprotein |
| | belonging to the M28 peptidase family. The protein acts |
| | as a glutamate carboxypeptidase on different alternative |
| | substrates, including the nutrient folate and the neuropeptide |
| | N-acetyl-1-aspartyl-1-glutamate and is expressed in a number of |
| | tissues such as prostate, central and peripheral nervous system |
| | and kidney. A mutation in this gene may be associated with |
| | impaired intestinal absorption of dietary folates, resulting in |
| | low blood folate levels and consequent hyperhomocysteinemia. |
| | Expression of this protein in the brain may be involved in a |
| | number of pathological conditions associated with glutamate |
| | excitotoxicity. In the prostate the protein is up-regulated in |
| | cancerous cells and is used as an effective diagnostic and |
| | prognostic indicator of prostate cancer. |
| EGFR | EGFR (epidermal growth factor receptor). The protein encoded |
| | by this gene is a transmembrane glycoprotein that is a member |
| | of the protein kinase superfamily. This protein is a receptor |
| | for members of the epidermal growth factor family. EGFR is a |
| | cell surface protein that binds to epidermal growth factor. |
| | Binding of the protein to a ligand induces receptor |
| | dimerization and tyrosine autophosphorylation and leads to cell |
| | proliferation. Mutations in this gene are associated with lung |
| | cancer. Diseases associated with EGFR include lung cancer, and |
| | paronychia, and among its related super-pathways are ErbB |
| | signaling pathway and Glioma |
| CXCR4 | CXCR4 (chemokine (C-X-C motif) receptor 4). This gene |
| | encodes a CXC chemokine receptor specific for stromal |
| | cell-derived factor-1. The protein has 7 transmembrane |
| | regions and is located on the cell surface. It acts with |
| | the CD4 protein to support HIV entry into cells and is also |
| | highly expressed in breast cancer cells. Mutations in this |
| | gene have been associated with WHIM (warts, |
| | hypogammaglobulinemia, infections, and myelokathexis) |
| | syndrome. Diseases associated with CXCR4 include whim |
| | syndrome, and intraocular lymphoma |
| IFITM3 | IFITM3 (interferon induced transmembrane protein 3). The |
| | protein encoded by this gene is an interferon-induced |
| | membrane protein that helps confer immunity to influenza |
| | A H1N1 virus, West Nile virus, and dengue virus. Diseases |
| | associated with IFITM3 include west nile virus, and |
| | pericardial tuberculosis. |

CD81 was highly enriched in the exosomial fraction confirming the quality of the preparation. A band of expected sized was detected with all antibodies, confirming the protein expression in these cells and are associated with the exosomial fraction.

### The OMV-Exosomes Formulation is Highly Immunogenic

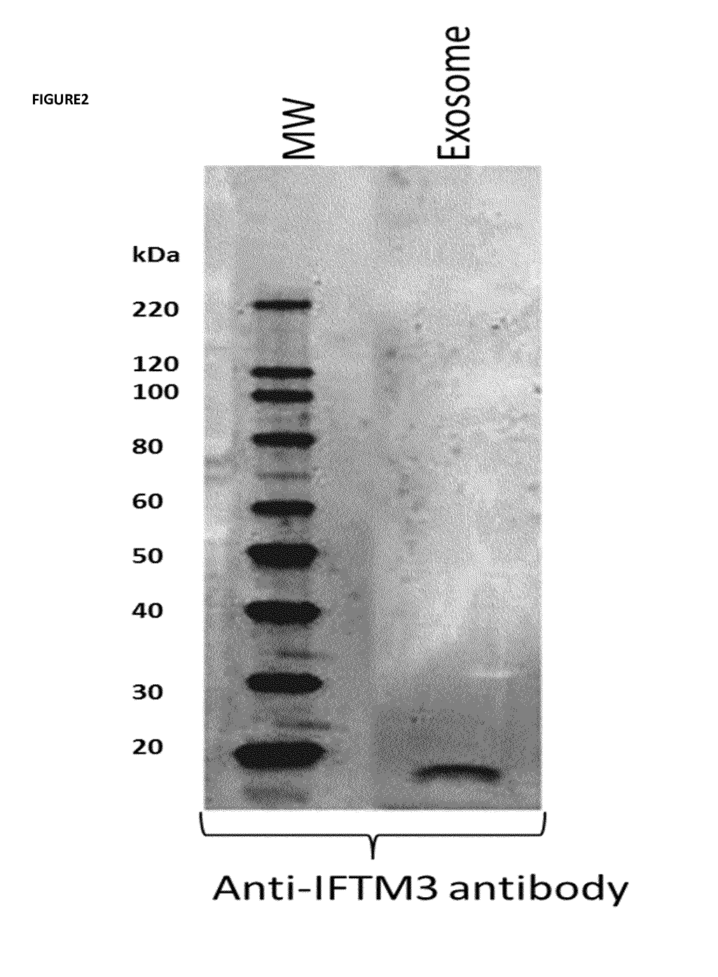

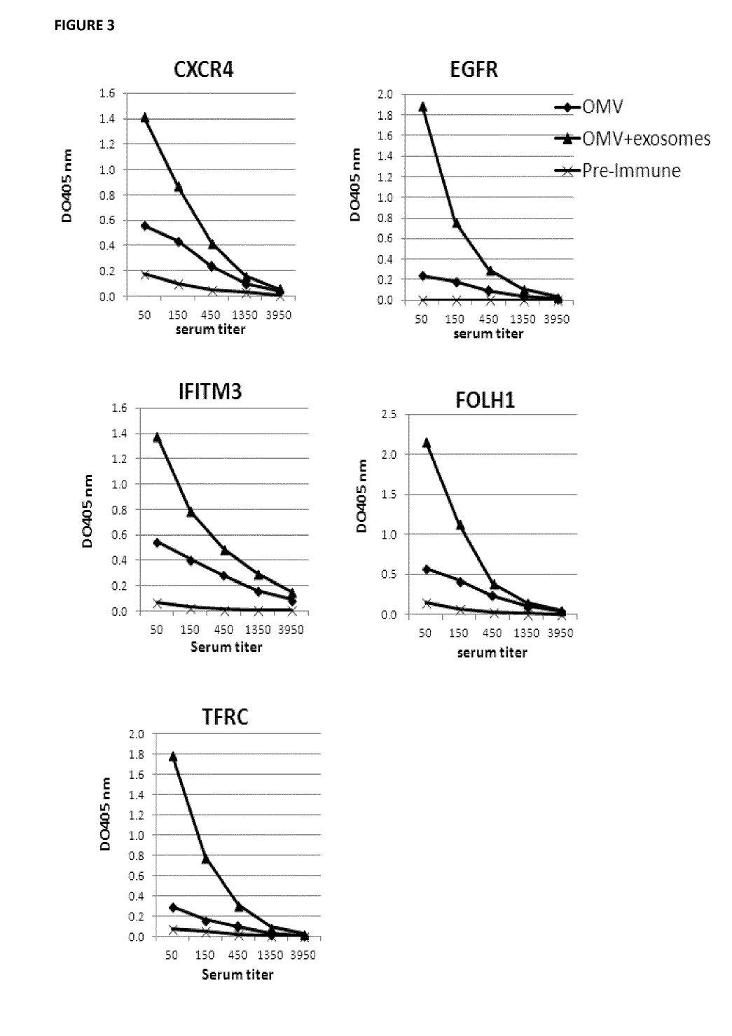

To verify the ability of the OMV-Exosome combination to elicit high antibody titers against exosomial proteins, CD1 mice were mice immunized with the combination OMV+exosomes (15+15 micrograms) and OMV (15 micrograms), formulated in Alum Hydroxide. Sera collected after the last immunization were pooled and analyzed by ELISA on plates coated with 5 recombinant proteins (see Table A). The proteins were selected for being exosomes associated and involved in a variety of human diseases (see Table A). As shown in FIG. 3, the combination OMV+exosomes induced high antibodies titers against 5/5 human proteins. Almost no antibodies were detected when mice were immunized with OMV alone, indicating that the antibody response elicited against each antigen by OMV+exosomes was specific, and not due to cross-reaction with OMV proteins. During the entire experiments mice did not showed any evident sign of toxicity or pain.

This piece of data indicates that the OMV-Exosomes formulation is safe and highly immunogenic, being able to elicit antibodies against all selected human proteins. Since the 5 exosomial proteins are known to be involved in different human pathologies, vaccines based on the OMV-Exosomes formulation could have wide applicability in the prevention or treatment of different diseases.

Antigen-Specific IgG Subclass Distributions in Sera from Immunized Mice

Adjuvants in combination vaccines can be used to reduce the immunization dose and number of injections, thereby decreasing undesired side effects (Dadan et al. (1998). Infect. Immun. 66:2093-20981998). Adjuvants potentiate or modulate the immune response of a particular antigen by creating a depot effect, targeting immune cells, or increasing the production of certain cytokines (Moingeon et al., (2001). Vaccine 19:4363-4372; Gupta et al (1995). Vaccine 13:1263-1276). Adjuvants can induce changes in the Th1-Th2 balance and thus in the antibody subclass generated. In mice, immunoglobulin G1 (IgG1) is associated with a Th2-like response, while a Th1 response is associated with the induction of IgG2a, IgG2b, and IgG3 antibodies (Germann et al, (1995). Eur. J. Immunol 25:823-829).

To understand whether immunization using the co-delivery of OMV with exosomes could shift the immune response balance toward a Th1 profile we compared the levels of IgG2a and IgG1 subclasses elicited in mice immunized with OMV+Exosomes, exosomes alone or OMV against the 5 selected proteins. Antigen specific IgG1 and IgG2a titers were measured in sera from mice immunized with the different formulations by ELISA, using anti-IgG1 and anti-IgG2a specific antibodies.

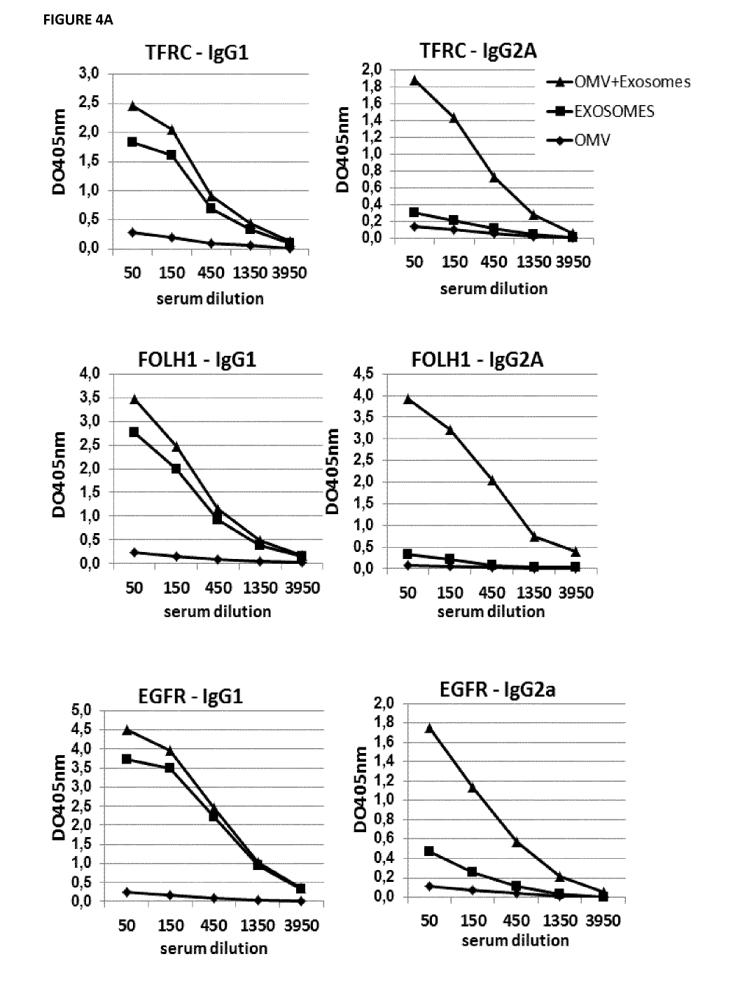

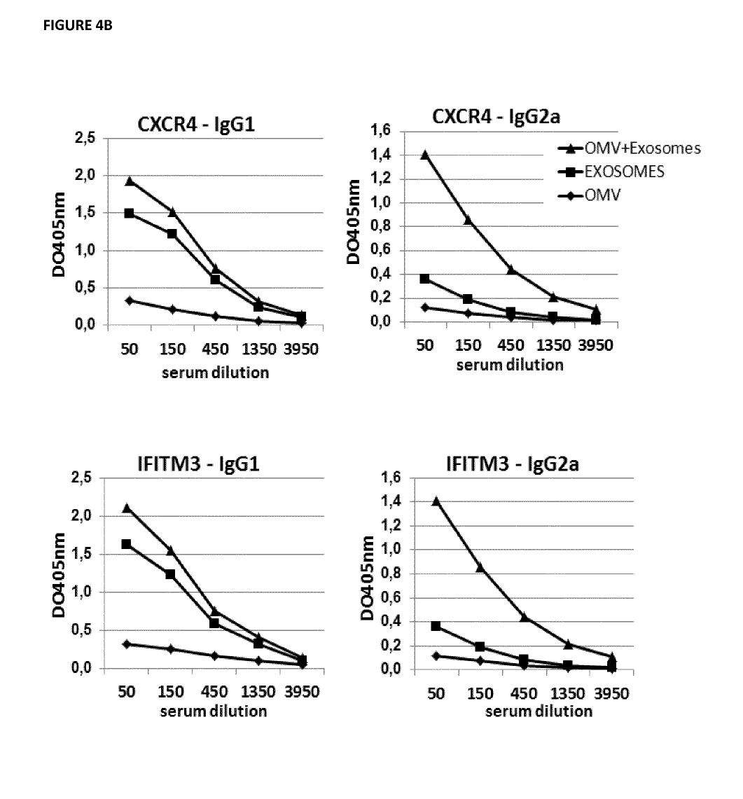

As shown in FIG. 4, immunization with OMV+exosomes and exosomes alone elicited a similar level of IgG. Conversely, OMV+exosomes were able to elicit a significantly higher antigen-specific IgG2a level than exosomes alone. This finding strongly indicates that the formulation OMV+exosomes is very effective in skewing the immune response in the Th1 direction.

It will be understood that the invention will be described by way of example only and modifications may be made whilst remaining within the scope and spirit of the invention.

### REFERENCES

[1] WO02/09643.
[2] Katial et al. (2002) Infect. Immun. 70:702-707.
[3] U.S. Pat. No. 6,180,111.
[4] WO01/34642.
[5] WO2004/019977.
[6] WO01/91788.
[7] WO2005/004908.
[8] WO2011/036562.
[9] WO2006/046143.
[10] WO00/26384.
[11] U.S. Pat. No. 6,531,131
[12] U.S. Pat. No. 6,645,503
[13] WO2004/015099.
[14] WO2006/081259.
[15] Deghmane et al. (2003) Infect Immun 71:2897-901.
[16] WO2006/024946
[17] Gennaro (2000) Remington: The Science and Practice of Pharmacy. 20th edition, ISBN: 0683306472
[18] Vaccine Design . . . (1995) eds. Powell & Newman. ISBN: 030644867X. Plenum.
[19] Berlanda et al. (2008). Mol Cell Proteomics 2008 March; 7(3):473-85.

1-15. (canceled)

16. An immunogenic pharmaceutical composition comprising an animal vesicle and a bacterial vesicle.

17. The immunogenic pharmaceutical composition of claim 16 wherein the animal vesicle comprises at least one disease-associated antigen.

18. The immunogenic pharmaceutical composition of claim 17 wherein the disease-associated antigen is selected from a tumor-associated antigen, a pathogen-associated antigen or a degenerative-disorder-associated antigen.

19. The immunogenic pharmaceutical composition of claim 18 wherein the tumor-associated antigen is selected from melan-A, Silv, carcinoembryonic antigen (CEA) and mesothelin.

20. The immunogenic pharmaceutical composition of claim 16 wherein the animal vesicle is an exosome or exosome-like vesicle.

21. The immunogenic pharmaceutical composition of claim 16 wherein the bacterial vesicle is an outer membrane vesicle, microvesicle or a native outer membrane vesicle.

22. The immunogenic pharmaceutical composition of claim 16 wherein the bacterial vesicle is modified by genetic recombination in the parent cell.

23. The immunogenic pharmaceutical composition of claim 16 wherein the animal vesicle and bacterial vesicle form a complex.

24. The immunogenic pharmaceutical composition of claim 23 wherein the complex is formed by fusion or by surface attachment of the two lipid bilayers.

25. The immunogenic pharmaceutical composition of claim 16 wherein the pharmaceutical composition is a vaccine composition.

26. A method for preparing a pharmaceutical composition, wherein the method comprises mixing an animal vesicle with a bacterial vesicle.

27. The method of claim 26 wherein the animal vesicle comprises at least one disease-associated antigen.

28. The method of claim 27 wherein the disease-associated antigen is selected from a tumor-associated antigen, a pathogen-associated antigen or a degenerative-disorder-associated antigen.

29. The method of claim 28 wherein the tumor-associated antigen is selected from melan-A, Silv, carcinoembryonic antigen (CEA) and mesothelin.

30. The method of claim 26 wherein the animal vesicle is an exosome or exosome-like vesicle.

31. The method of claim 26 wherein the bacterial vesicle is an outer membrane vesicle, microvesicle or a native outer membrane vesicle.

32. The method of claim 26 wherein the bacterial vesicle is modified by genetic recombination in the parent cell.

33. The method of claim 26 wherein the animal vesicle and bacterial vesicle form a complex.

34. The method of claim 32 wherein the complex is formed by fusion or by surface attachment of the two lipid bilayers.

35. The method of claim 26 wherein the pharmaceutical composition is a vaccine composition.
